(19) United States
(12) Patent Application Publication
(10) Pub. No.: US 2005/0220713 A1
(43) Pub. Date: Oct. 6, 2005

(54) Masticatory function test wax cube, and method and apparatus for producing the same

(75) Inventors: Takashi Ohyama, Kanagawa (JP); Hiroyuki Takahashi, Tokyo (JP); Mutsumi Nakata, Miyagi (JP); Hideaki Mizuma, Miyagi (JP); Rui Fujiwara, Miyagi (JP)

Correspondence Address: MCDERMOTT WILL & EMERY LLP, 600 13TH STREET, N.W., WASHINGTON, DC, 20005-3096, US

(73) Assignees: INOUE ATTACHMENT CO., LTD.; Takashi OHYAMA

(21) Appl. No.: 11/062,828

(22) Filed: Feb. 23, 2005

(30) Foreign Application Priority Data
Feb. 26, 2004 (JP) 2004-052496

(51) Int. Cl. A61K 49/00

(52) U.S. Cl. 424/9.6; 424/49

(57) ABSTRACT

The invention provides a masticatory function test wax cube in which two-color colored waxes are in close contact with each other with no gap, and the two-color colored waxes are evenly connected to each other with sufficient strength, and a method and apparatus for producing the masticatory function test wax cube. A wax cube 1 with a pseudo-checkered pattern is produced by injection molding a first-color colored wax 2 to form integrally a first-color wax cube in work and then injecting and filling a second-color colored wax 3 into an empty space of the wax cube in work.

### BACKGROUND OF THE INVENTION

1. Field of the Invention

The present invention relates to a masticatory function test wax cube used for evaluating a masticatory performance at occlusion of upper and lower teeth of a human in a simple and convenient manner in the field of the dentistry; and a method and apparatus for producing the masticatory function test wax cube.

2. Description of the Related Art

A masticatory function of a human, i.e. occlusion of upper and lower teeth is composed of a combination of complicated movements and workings, and it is said that the masticatory performance is closely associated with a human health condition. Conventionally, various methods have been proposed in order to objectively evaluate the human masticatory performance. However, the every conventional method has following problems. That is, the procedures of evaluation are complicated, the examining processes are troublesome, the evaluation result lacks objectivity, only particular data can be obtained, and the examination apparatus is expensive. Examples of the conventional simple method to evaluate a human masticatory performance include a method of measuring a sugar content remaining in the chewed gum after a subject chews gum by a predetermined times, and a method of checking a degree of crushing by causing a subject to bite raw rice or peanut. However, in these methods, it is difficult that the masticatory performance is evaluated qualitatively and in detail. Further, the methods do not have sufficient reliability. In addition, a method has also been proposed, in which an alpha substituted grain containing a fluorescent agent is used as test food, saliva in a mouth cavity is taken as a sample after chewing the test food, a piece of filter paper is spotted with a certain amount of the sample, and a measurement is performed by irradiation of a ultraviolet ray (see Japanese Patent Application Laid-Open (JP-A) Nos. 6-167452 and 6-254107). However, in the method, there is a problem in that the method can only determine total masticatory ability and cannot determine the detail masticatory performance. The measurement of occlusal force with a strain gage load transducer has also been proposed (see JP-A-8-215223). However, the teeth of humans infinitely differ from one another in a shape, a size and orientation, or a natural tooth or an artificial tooth. It is not, therefore, always said that the accurate measurements are performed in all the cases, and also there is the problem in that only one aspect of the masticatory function can be measured by this method.

On the other hand, there has been proposed recently a system in which the masticatory performance is simply and objectively evaluated with a masticatory function test wax cube 1 as a text food, wherein each of six surfaces showed a pseudo-checkered pattern by red and green colored waxes as schematically shown in FIG. 1 (refer to Journal of Oral Rehabilitation 30, 68-73, Blackwell Publishing Ltd. (2003)). The wax cube used in this evaluation method has the dimensions of 12 mm×12 mm×12 mm. The wax cube 1 includes slender red and green small rod-shaped wax bodies 2 and 3 having the identical dimensions of 2 mm×2 mm×12 mm. In the wax cube 1, each three of rod-shaped wax bodies 2 and 3 are alternately arranged in one plane, and each three of rod-shaped wax bodies 2 and 3 are arranged in the same way while rotated by 90 degrees in the next stage. The arrangements are repeated in six stages to form a cubic wax by attaching the rectangular parallelepiped waxes to each other. The attachment between the rod-shaped wax bodies is made by physical bonding power and adhesion between the waxes when the wax is softened. The aforementioned size of the wax cube used for examining masticatory function has been determined as suitable only by results of various studies and trials. Therefore, the size of the wax cube is not limited to the above-described size. However, from a viewpoint of insistency of the measurement results, it is recommended that the wax cube having the above-described size should be used. The colors used in the above-described wax cube have been conventionally selected only because the combination of red and green is tentatively preferable from a viewpoint of image analysis. Therefore, the color combination is not limited to the combination of red and green. For example, a combination of red and blue can be cited as an example of other color combinations. However, because the examination data has been recorded by using the wax cube having the combination of red and green as a test food, it is desirable currently that the combination of colors on the wax cube used is red and green in order to maintain the consistency of the measurement data.

The method of evaluating the masticatory function with the wax cube will be described referring to FIG. 2. After the wax cube 1 shown in FIG. 1, in which the red and green waxes are arranged in a pseudo-checkered pattern, is heated to 37° C., the wax cube 1 is put in a mouth cavity, the wax cube is chewed by the predetermined times, and the wax cube is taken out from the mouth cavity to make a test sample 10. Then, the image of the sample 10 is taken with a CCD color camera 11, and the image data is transmitted to an image analyzer 12. From the image data, the data of the sample such as a projection area, a width, a length, a thickness distribution (for example an area in which a thickness of the test sample is not lower than 50 μm and an area in which a thickness of the test sample is lower than 50 μm), a color mixing state, and a color distribution state (area of remaining red and green regions) of the test sample 10 is measured. Then, data analysis is performed on the basis of the measurement data with a personal computer 13, a mixing ability coefficient is computed from a given relational expression, and the masticatory performance of the subject is evaluated from the mixing ability coefficient.

However, since the wax cube has the complicated structure of pseudo-checkered pattern as described above, the complicated process is conventionally required in order to produce the wax cube. Namely, in order to produce the wax cube, a wax and a coloring agent as raw materials are heated together to dissolve the wax, a dissolved liquid wax is mixed to attach the wax and the coloring agent sufficiently and disperse uniformly the coloring agent in the wax. Then, the colored wax solution is run into a die to produce a slender rod-shaped colored wax body having the dimensions of 2 mm×2 mm×12 mm. In production of the colored wax body, there is used a flat-shaped die which is formed by installing, in a die plate, a insert die of 12 mm in width having many rectangular grooves of, for example, 2 mm in depth, 2 mm in width, and 12 mm in length in parallel so as to be orthogonal to a lengthwise direction of the insert die. After the colored wax solution is run into the die, a flat plate (upper plate) is put on the die, and the die and the upper plate are pressurized at pressure ranging, for example, from 55 to 60 Kg by a press. The upper plate is removed after releasing the pressure, a wax body-forming portion in the die is sufficiently cooled, e.g. in running water, and then excess wax adhering to the die is removed. Thereafter, when the insert die is taken out from the die plate, many rod-shaped wax bodies are formed in the grooves and the colored wax surface of 2 mm×2 mm is exposed in a side face of the insert die. The slender rod-shaped colored wax bodies having the dimensions of 2 mm×2 mm×12 mm are obtained by taking out the wax body from the groove of the insert die. Many rod-shaped wax bodies can be produced by repeatedly conducting these processes. Using another-color colored wax similarly produces another color rod-shaped wax body. The following methods are adopted in order to make the wax cube using the rod-shaped wax bodies, namely, a method in which each three of red and green rod-shaped waxes 2 and 3 are alternately arranged to make a layer consisting of six rod-shaped waxes, the layers are laminated to the six stages while the orientation of the layer is changed, and an assembly of 36 rod-shaped waxes is pressurized and moderately heated in the die once to attach the neighboring waxes to each other; and a method in which each three of wax bodies 2 and 3 having the identical dimensions of 2 mm×2 mm×12 mm are alternately arranged while the colors of the neighboring wax bodies are different from each other, a red and green stripe flat-shaped wax body having the dimensions of 12 mm×12 mm×2 mm is temporarily formed by pressurizing and moderately heating the wax bodies once to attach the neighboring waxes to each other, six layers of the flat-shaped wax bodies are laminated by arranging the flat-shaped wax body on the flat-shaped wax body formed in the same way while the stripes of the neighboring two flat-shaped wax bodies are orthogonal to each other, and the neighboring flat-shaped wax bodies are pressurized and moderately heated again to attach the neighboring flat-shaped wax bodies to each other.

Thus, since the conventional wax cube is produced by manually assembling the wax cube one by one, it takes a lot of trouble and a longtime to produce the wax cube and the conventional wax cube is poor in productivity. Further, in production of the wax cube, it is difficult to obtain the 36 small rod-shaped wax bodies having the same size, and there is somewhat of difference in the individual rod-shaped wax body. Therefore, it is difficult to produce a wax cube that is evenly attached with sufficient strength and with no gap, and when a skill of an assembly worker is not high, a frequency of generation of a defective product becomes high. As a result there is the problem in that the good wax cube cannot be produced at a low price.

### SUMMARY OF THE INVENTION

In order to solve the above-described problems, an object of the invention is to provide a method for producing a masticatory function test wax cube wherein each of two colored waxes constituting the wax cube is integrally formed, the two colored waxes are in close contact with each other with no gap, the two-color colored waxes are evenly attached to each other with sufficient strength, and the masticatory function test wax cube having good quality is produced with good productivity without generating the defective product.

Another object of the invention is to provide an apparatus for producing a masticatory function test wax cube with which the masticatory function test wax cube having good properties is produced with good productivity without generating the defective product.

Still another object of the invention is to provide a masticatory function test wax cube wherein the two colored waxes produced by the above-described producing method are in close contact with each other and each of the two colored waxes is integrally formed.

The invention relates to a method for producing a masticatory function test wax cube, an apparatus for producing a masticatory function test wax cube, and a masticatory function test wax cube described in the following items.

(1) A method for producing a two-color masticatory function test wax cube with a pseudo-checkered pattern used for evaluating a masticatory performance, wherein a first-color colored wax is injection-molded to produce integrally a first-color wax portion of the wax cube and then a second-color colored wax is injected and filled into empty space of the wax cube to produce a wax cube consisting of two-color colored waxes with a two-color pseudo-checkered pattern.

(2) A method for producing a masticatory function test wax cube described in the item (1), wherein the colored wax constructing the wax cube comprises a wax and a coloring agent.

(3) A method for producing a masticatory function test wax cube described in the item (1) or (2), wherein the wax comprises a paraffin wax and another wax that imparts adhesive properties to the paraffin wax, and the coloring agent comprises a crayon pastel.

(4) A method for producing a masticatory function test wax cube described in any one of the items (1) to (3), wherein colored wax of a raw material used for the injection molding or injection is supplied in powder form to an injection molding apparatus or an injection apparatus.

(5) A method for producing a masticatory function test wax cube described in any one of the items (1) to (3), wherein the colored wax of a raw material used for the injection molding or injection is produced as a homogeneous colored wax solution by melting and mixing a wax and a coloring agent and then supplied to an injection molding apparatus or an injection apparatus in the condition cooled down to 40° C. to 50°.

(6) A method for producing a masticatory function test wax cube described in any one of the items (1) to (5), wherein an injection molding temperature or an injection temperature of the colored wax ranges from 40° C. to 45° C.

(7) A method for producing a masticatory function test wax cube described in any one of the items (1) to (6), wherein, when a first-color injection molded article is produced with the colored wax, a slide core is arranged around the molding die to produce the injection molded article having a multi-stage structural pseudo-checkered pattern.

(8) A method for producing a masticatory function test wax cube described in the item (7), wherein the first-color injection molded article that is a work-in-process wax cube is produced and then taken out from a first molding die and after the first-color injection molded article is placed into a second molding die, the second-color colored wax is injected into the empty space of the first-color injection molded article housed in the second molding die to produce a wax cube with a two-color pseudo-checkered pattern.

(9) A method for producing a masticatory function test wax cube described in the item (7), wherein after the first-color injection molded article that is a work-in-process wax cube is produced, a slide core pin is pull out from the inside of the molding die without taking out the first-color injection molded article from the molding die, and then the second-color colored wax is injected into the empty space of the first-color injection molded article in the molding die to produce a wax cube with a two-color pseudo-checkered pattern.

(10) A method for producing a masticatory function test wax cube described in the item (7), wherein a die is formed by moving of four slide cores having slide core pins in clamping the die, a first-color injection molded article is produced by injecting a first-color colored wax into the die, the slide cores are backed by a die opening operation, then the first-color injection molded article is moved together with the slide core to a second-color molding portion, the first-color injection molded article is housed in a second die in which the injection molded article can be housed, and second-color colored wax is injected into an empty space of the first-color injection molded article to produce a wax cube with a two-color pseudo-checkered pattern.

(11) A method for producing a masticatory function test wax cube described in any one of the items (1) to (10), wherein the masticatory function test wax cubes including two-color colored waxes are placed in each concave portion of a backing sheet for packing, and the each concave portion is sealed with a package film.

(12) An apparatus for producing a first-color injection molded article of a masticatory function test wax cube, comprising;
nine substantially square holes which are pierced through a side wall of a die having outside surfaces orthogonal to each other from the outside surfaces of the die toward the cubic concavity in the die, the nine square holes being made in each face,
a slide core provided with nine rod-shape core pins having a substantially square cross-sectional area so as to pierce through the holes,
a slide core-moving device for moving the slide core, and
an injection nozzle for injecting a first-color colored wax into a concavity of the die.

(13) An apparatus for producing a first-color injection molded article of a masticatory function test wax cube described in the item (12), wherein the core pin is attached to a core pin attaching portion of the slide core, and a core pin attaching portion of the slide core can be moved by an air cylinder.

(14) An apparatus for producing a first-color injection molded article of a masticatory function test wax cube described in the item (12) or (13), wherein the core pine is attached to a core pin attaching portion of the slide core, and a hole for piecing an angular pin is formed in the core pin attaching portion of each slide core, and the slide core can be moved corresponding to inserting into or drawing out the angular pin with the hole.

(15) An apparatus for producing a masticatory function test wax cube, comprising;
a jig fixed to a backing plate, which has a surface for positioning the first-color injection molded article of a masticatory function test wax cube that is a work-in-process wax cube and forming a side face of a concave portion in which the wax cube injection molded article is housed, a runner and a gate,
a slider that forms a concavity, in which the wax cube injection molded article is to be housed, with the jig, and
an injection nozzle that injects a colored wax into the concavity formed by the jig and the slider.

(16) An apparatus for producing a masticatory function test wax cube, comprising;
a fixed plate having two square holes, one of which is a position to form a wax cube injection molded article with a first-color colored wax and another of which is an injection position to inject a second-color colored wax into a die,
a die-fixing plate that is provided below the fixed plate, and is able to revolve and position, in the hole, one of dies fixed to the die-fixing plate by moving the die-fixing plate upward,
the dies which are fixed to the die-fixing plate and through side walls of which holes for respectively inserting a slide core pin are formed,
slide cores that are arranged in each side of the hole and have the slide core pins,
slide core moving means for moving the slide core, and
colored wax-filling cylinders that are movably arranged above the each hole.

(17) An apparatus for producing a masticatory function test wax cube in which a filling cylinder for injecting a first-color colored wax into a die and a filling cylinder for injecting a second-color colored wax into the die are connected in parallel, wherein
the die has a square shape,
nine holes into which core pins are inserted are made in each side surface of the die,
slide cores in which the core pins are arranged at a position corresponding to each hole into which core pins are inserted, are provided opposing to the side surface of the die,
slide core moving means that cause the core pins to be inserted into the die when the first-color colored wax is injected into the die and cause the core pins to be pulled out from the inside of the die after the injection of the first-color colored wax is completed is provided,
the die is moved to a second-color colored wax injection position by conveying means after the injection of the first-color colored wax is ended, and
a second-color colored wax is injected into the die after a leading end surface of the core pin of the slide core is moved to a position where the leading end face of the core pine forms a part of an inner wall surface of a concavity in the die for forming a wax cube.

(18) An apparatus for producing a masticatory function test wax cube in which a filling cylinder for injecting a first-color colored wax into a first die and a filling cylinder for injecting a second-color colored wax into a second die are connected in parallel,
the first die includes four slide cores in which each core pin is formed inward,
core pin-attaching surfaces of the slide cores and core pines are arranged such that a square-shaped concavity is formed by the core pin-attaching surfaces of the slide cores and a multi-stage pseudo-checkered pattern block is formed by the core pins when the slide cores are moved to the center on the same plane,
the first-color colored wax is injected to form a first-color injection molded article for a masticatory function test wax cube (work-in-process wax cube) in the first die while the concavity is formed,
the first die is opened and the core pins are pull out from the inside of the injection molded article for the wax cube,
the injection molded article for the wax cube is conveyed with the first die to a second-color colored wax injection position, and
the masticatory function test wax cube is produced by injecting the second-color colored wax into a cavity portion of the first-color injection molded article, which is formed by pulling out the core pins, by the second-color filling cylinder while the wax cube injection molded article is covered with second die at the second-color colored wax injection position, thus the masticatory function test wax cubes being prepared.

(19) A colored wax-powdering apparatus for use in production of a masticatory function test wax cube, wherein the apparatus comprises;
an extruding device for extruding a colored wax,
a perforated die having many holes and being attached to a leading end portion of the extruding device, and
a pulverization net oscillatably attached to an outer peripheral surface of the perforated die.

(20) A masticatory function test wax cube including two-color colored waxes, which is formed by injection-molding integrally a first-color colored wax portion, and then injecting a second-color colored wax into an empty space of the wax cube to fill the empty space with the second-color colored wax.

Advantageous effects are shown by the preset invention as following.

The masticatory function test wax cube, wherein two colored waxes constituting the wax cube are in close contact with each other with no gap and are attached to each other with even and sufficient strength, can be obtained with higher productivity by the method for producing a masticatory function test wax cube described in the item (1).

The wax cube used suitably in the masticatory functional test can be produced by the method for producing a masticatory function test wax cube described in the item (2).

In the method for producing a masticatory function test wax cube described in the item (3), there is an effect that no separation of the wax cube occurs by use of a paraffin wax and another wax for imparting adhesive properties to the paraffin wax when the produced masticatory function test wax cube is chewed in the examination.

According to the method for producing a masticatory function test wax cube described in the item (4), the colored wax can be preserved at the state where the coloring agent is evenly dispersed, and the injection molding of the first-color colored wax or the injection of the second-color colored wax can be performed at an arbitrary time. Further, there is an effect that after the colored wax solution is solidified and powdered, the powdered wax can be supplied as a raw material as a stable quality as a whole by stirring the powder, even if an uneven state is generated in the coloring within the same production batch or among different production batches.

Production of the colored wax and injection molding by the colored wax or injection of the colored wax can be continuously performed by the method for producing a masticatory function test wax cube described in the item (5).

The high-quality masticatory function test wax cube can be produced with higher productivity by the method for producing a masticatory function test wax cube described in the items (6) and (7).

According to the method for producing a masticatory function test wax cube described in the item (8), mass production of the wax cube can be achieved with a simple apparatus.

The high-quality masticatory function test wax cube can be produced with higher productivity by the method for producing a masticatory function test wax cube described in the items (9) and (10).

According to the method for producing a masticatory function test wax cube described in the item (11), the produced masticatory function test wax cube can be blocked (sealed) from ambient air, and the wax cube can be hygienically preserved and handled.

The high-quality work-in-process masticatory function test wax cube can be produced with higher production efficiency by the apparatus for producing a work-in-process masticatory function test wax cube described in the items (12) to (14).

According to the apparatus for producing a masticatory function test wax cube described in the item (15), the colored wax that has the color different from the work-in-process wax cube can be injected into the empty space of the work-in-process wax cube with higher productivity to densely fill the empty space with the colored wax. Therefore, the two-color waxes are in close contact with each other with no gap, and the high-quality wax cube in which the two colored waxes are attached to each other with even and sufficient strength can be mass-produced in the even quality.

The high-quality masticatory function test wax cube including two colored waxes can be continuously produced by the apparatus for producing a masticatory function test wax cube described in the items (16) to (18).

According to the colored wax-powdering apparatus described in the item (19), the high-quality powdery colored wax can be produced with a simple apparatus.

According to the masticatory function test wax cube described in the item (20), each of the two colored waxes is integrally formed respectively, the two colored waxes are in close contact with each other with no gap, and the two colored waxes are attached to each other with even and sufficient strength. Therefore, problems such that no breakage of the wax cube occurs before the examination, and the wax cube has the even quality, so that the disadvantage such as the separation of the two colored waxes never occurs in the masticatory function test, and the test can be performed always under a constant condition.

Then, the invention will be described more specifically below.

The wax cube produced in the invention is used for examining the masticatory performance. Therefore the various properties are required for the wax cube as following. Namely, the wax cube is harmless to humans, the wax cube has proper softness in an oral cavity during the masticatory function test, the wax cube has an appropriate strength in being chewed, the two colored waxes are not separated from each other by the chewing, and the like.

Such a masticatory function test wax cube is basically produced using a wax and a coloring agent. Examples of the wax used for the production of the wax cube include a paraffin wax that is a typical wax material used for formation of a wax dam in a dental laboratory and another wax by which properties of the paraffin wax are modified to impart the preferable properties as the wax for examining the masticatory function. In the invention, it is preferable to use these waxes at the same time. As the paraffin wax used for the wax cube of the invention, a paraffin wax having a melting point from about 55° C. to about 65° C. and a penetration rate from about 10 to 15 at 25° C. is preferred. The preferable example of the paraffin wax used for the wax cube of the invention is PARAFFIN WAX 140 among standard products of PARAFFIN WAX 115, 120, 125, 130, 135, 140, 150 and 155 (trade name, produced by NIPPON SEIRO CO., LTD). PARAFFIN WAX 140 has a melting point of 61° C., a viscosity of 4.1 mm²/s at 100° C., penetration rates of 11 at 25° C. and 17 at 35° C., densities of 0.921 at 25° C. and 0.800 at 100° C., and an adhesive strength (AI/paper) of 196 mn/cm at 20° C.

As an example of the wax that is preferably used with the paraffin wax to act as a modifying agent for modifying flexibility and adhesion of the paraffin wax, Hi-Mic 1045, 1090 and 1080 (trade name, produced by NIPPON SEIRO CO., LTD) that are of a microcrystalline wax can be cited. In Hi-Mic 1045, 1090 and 1080, Hi-Mic 1045 is preferably used. Hi-Mic 1045 has a melting point of 70° C., a viscosity of 15.8 mm²/s at 100° C., a penetration rate of 30 at 25° C., densities of 0.921 at 25° C. and 0.800 at 100° C., and an adhesive strength (AI/paper) of 196 mn/cm at 20° C. In this case, the amount of the modifying wax for improving the properties of the paraffin wax depends on the melting point, the penetration rate, and the adhesive strength of the modifying wax used. Generally, the modifying wax ranging from about 50 to 200 parts by weight is used with respect to 100 parts by weight of the paraffin wax. When the PARAFFIN WAX 140 and Hi-Mic 1045 are used as the paraffin wax and the modifying wax, the amount of Hi-Mic 1045 used is preferably from 80 to 120 parts by weight to 100 parts by weight of the PARAFFIN WAX 140, more preferably 100 parts by weight to 100 parts by weight of the PARAFFIN WAX 140.

The coloring agent is not particularly limited, as long as the coloring agent is a dye or pigment that has good compatibility with waxes such as a paraffin wax and is harmless to humans. Particularly, commercially available crayon pastel in which a dye or pigment has been already mixed with a wax is preferably used for the purpose of the invention. For example, when the two-color wax cube including red and green is produced, it is preferable to use the red crayon pastel as a coloring agent for the red colored wax and the green crayon pastel and yellow-green crayon pastel as a mixture as a coloring agent for the green colored wax. When the crayon pastel is used as the coloring agent, the amount of the crayon pastel is preferably 2 to 10 parts by weight, more preferably from 3 to 5 parts by weight relative to 100 parts by weight of the paraffin wax and the modifying wax in total.

### BRIEF DESCRIPTION OF THE DRAWINGS

FIG. 1 is a perspective view of a masticatory function test wax cube.

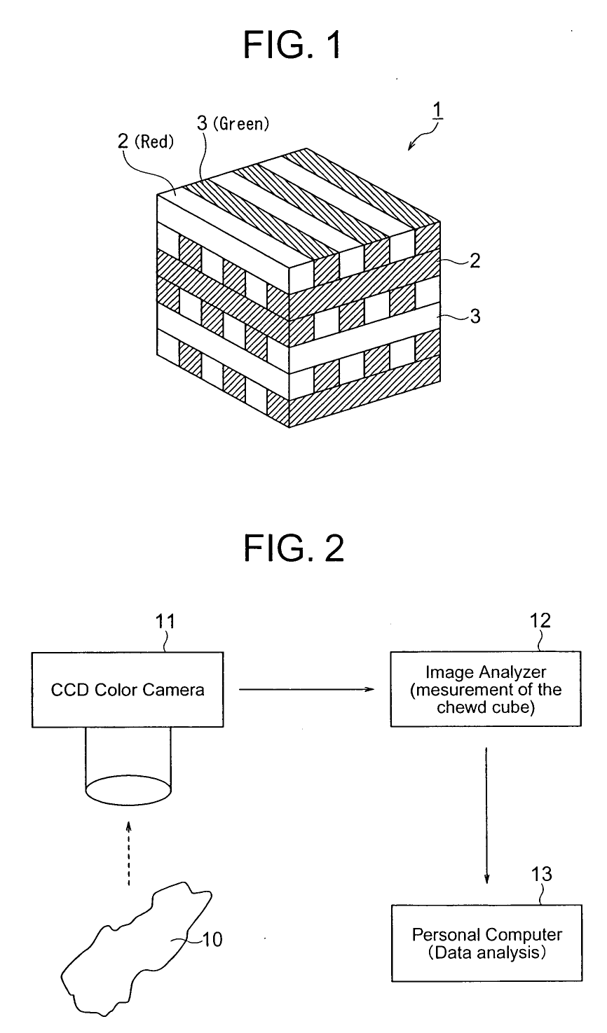

FIG. 2 is an explanatory view showing a method of evaluating a masticatory performance with a sample that is obtained by chewing a masticatory function test wax cube.

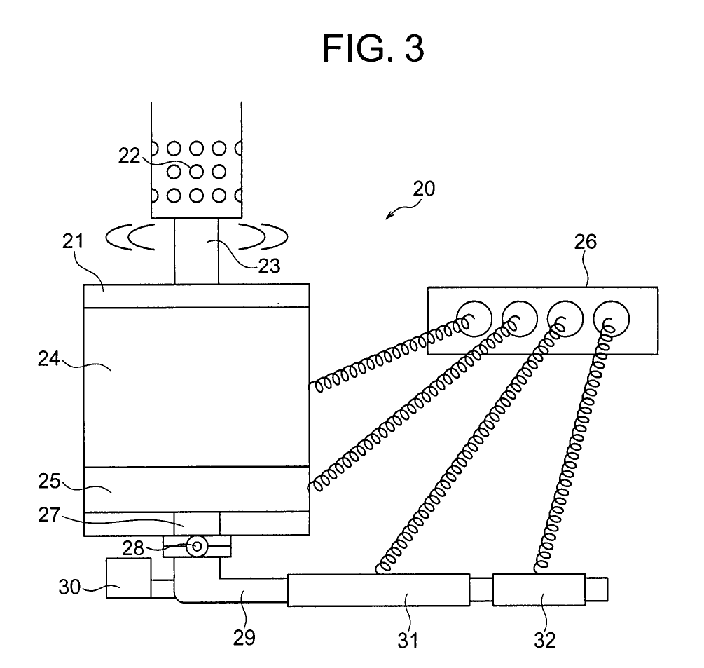

FIG. 3 is an external view of a wax melting and stirring apparatus.

FIG. 4 is an external view of a wax powdering apparatus.

FIG. 5 is a development view (hexahedral view) of a work-in-process wax cube.

FIG. 6 is a front view of an injection molding apparatus.

FIG. 7 is a plan view of a cavity-retainer plate and a movable retainer plate of a die used for producing the work-in-process wax cube.

FIG. 8 is a sectional view of a slide core.

FIG. 9A is a bottom view of the movable retainer plate (movable die plate) on which angular pins are provided, and FIG. 9B is a plane view showing the cavity-retainer plate opposing to the movable retainer plate of FIG. 9A.

FIG. 10 is a plane view of the cavity-retainer plate that is used for filling empty space of the green work-in-process wax cube with a red colored wax.

FIG. 11 is a front view of an apparatus for producing masticatory function test wax cube continuously and automatically.

FIG. 12 is a plan view of a fixed plate of an apparatus for continuously and automatically producing masticatory function test wax cube showed in FIG. 11.

FIG. 13 is a view of a shape of a die fixing plate and a positional relationship between the fixed plate and the die fixing plate in the apparatus for continuously and automatically producing masticatory function test wax cube showed in FIG. 11;

FIG. 14A is an enlarged view of a slide core in a filling portion of a first-color colored wax on the fixed plate of the apparatus for continuously and automatically producing masticatory function test wax cube showed in FIG. 11, and FIG. 14B is an enlarged view showing a slide core in a filling portion of a second-color colored wax.

FIG. 15 is a plan view of another masticatory function test wax cube continuously and automatically producing apparatus;

FIG. 16 is a perspective view showing a packing machine for packaging the masticatory function test wax cube.

FIG. 17 is a perspective view showing the packaged masticatory function test wax cube.

### DETAILED DESCRIPTION OF THE PREFERRED EMBODIMENTS

Preferred embodiments of a method and apparatus for producing a masticatory function test wax cube of the invention will specifically be described below.

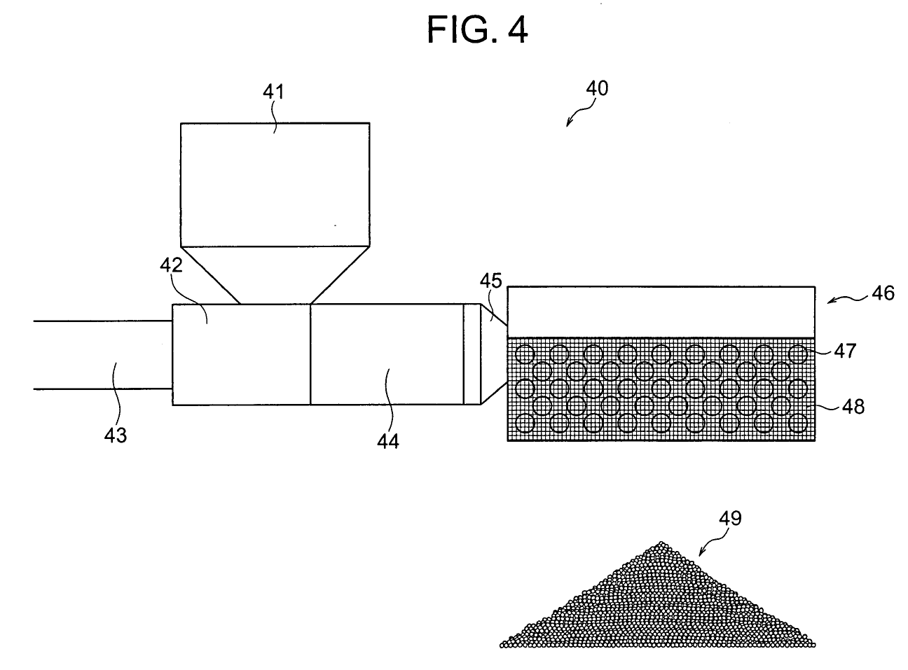

In order to produce the masticatory function test wax cube of the invention, it is necessary to produce a colored wax. So a method and apparatus for producing the colored wax will first be described referring to FIG. 3. The apparatus for producing the colored wax shown in FIG. 3 and the method for producing the colored wax using the apparatus for producing the colored wax (colored wax producing apparatus) are illustrates only by way of examples for explaining the method for producing the colored wax of the invention, and the apparatus and method for producing the colored wax of the invention are not limited to ones showed in FIG. 3.

In FIG. 3, reference numeral 20 represents a wax-melting and stirring apparatus, and a wax-melting vessel 21 is arranged in the wax-melting and stirring apparatus 20. A stirring screw 23 connected to a drive motor 22 is inserted into the vessel 21 from the upper position. Heating devices such as a band heater 24 and a circular heater 25 are attached to an outer surface of the vessel 21. A temperature control box 26 controls the heating devices so that the vessel 21 becomes a constant temperature suitable for melting and stirring the wax and the coloring agent. An exhaust port 27 is provided in a lower portion of the vessel 21, and a drain 28 is attached to the exhaust port 27 in order to discharge the melted and mixed colored wax. A material-transporting pipe 29 in which a transport screw is provided is connected to the drain 28. The heating device such as band heaters 31 and 32 is also attached to the outer surface of the material-transporting pipe 29 and the temperature control box 26 conducts the temperature control of the heating device to stepwise decrease the temperature of the colored wax transported through the material transport pipe 29.

First, the method for producing the green colored wax by the wax melting and stirring apparatus 20 shown in FIG. 3 is described concretely because the red and green waxes are historically used to produce the masticatory function test wax cube.

A wax and a crayon pastel used as a coloring agent are weighed in order to produce the green colored wax. In this case, 100 parts by weight of the wax in total (50 parts by weight of PARAFFIN WAX 140 and 50 parts by weight of HI-WAX 1045) are used. As the green coloring agent, commercially available green and yellow-green crayon pastels (trade name; Sakura Cray-Pas, manufactured by SAKURA COLOR PRODUCTS CORP.) are used, and the 8 parts by weight of crayon pastels in total (each 4 parts by weight of green and yellow-green crayon pastels) are used relative to the 100 parts by weight of the wax in total. After the wax and the crayon pastels are weighed, the predetermined amounts of wax and crayon pastel are loaded into the wax-melting vessel 21 of the wax-melting and stirring apparatus, respectively. In the wax-melting and stirring apparatus, the vessel is controlled at a predetermined temperature by the band heater 24 and the circular heater 25. When the wax is melted to a certain degree, the stirring screw 23 stirs the wax, and the wax is further heated. At this point, there occur problems that the wax is volatilized when the heating temperature of the wax-melting and stirring apparatus 20 is too high, and that the melting speed of the wax is slow and the wax and the coloring agent are not sufficiently mixed when the heating temperature of the wax-melting and stirring apparatus 20 is too low. Therefore, the heating temperature of the wax is set in the temperature range in which the above troubles are not generated. Although the heating temperature depends on the type of wax, it is preferably set at about 70° C. to 85° C. Usually about 10 minutes to 15 minutes are adequate for further stirring the wax and the crayon pastel after the wax and the crayon pastels are liquefied. The stirring time is not particularly limited, and the stirring time longer or shorter than that of the above-described range can be set as long as the finally evenly colored wax is obtained. When the wax and the coloring agent are sufficiently mixed together and the coloring agent is wetted sufficiently with the wax to evenly melt and color the wax in the vessel 21, the liquid colored wax is discharged to the material-transporting pipe 29 through the drain 28 that is provided in the discharge portion in the bottom portion of the apparatus and connected to the material-transporting pipe 29. The transport screw is arranged in the material-transporting pipe 29, and the liquid colored wax is transported through the material-transporting pipe 29 by rotating the screw with a screw drive motor 30. At this point, the band heaters 31 and 32 wound around the material-transporting pipe 29 control the temperature of the liquid colored wax. The liquid colored wax having the temperature of about 70° C. to 85° C. at first is controlled so that the temperature of the liquid colored wax is decreased to the range from 40° C. to 45° C. when the liquid colored wax is discharged from the material-transporting pipe 29. The discharged colored wax that is controlled in the above temperature range shows a creamy or grease state.

In addition, in order to produce the red colored wax, there may be used, for example, 50 parts by weight of PARAFFIN WAX 140, 50 parts by weight of HI-WAX 1045 as with the production of the green colored wax and 8 parts by weight of a red crayon pastel (tradename; Sakura Cray-Pas, manufactured by SAKURA COLOR PRODUCTS CORP.) as the coloring agent.

The colored wax discharged from the wax-melting and stirring apparatus is used as a raw material for producing a wax cube in the injection molding process with the colored wax or the injection process with the colored wax, which is the subsequent process. It is possible that the colored wax produced in the colored wax producing process is directly used as the raw material for the injection molding process or the injection process, and it is also possible that the solidified colored wax is used later as the raw material for the injection molding process or the injection process after the colored wax was solidified once.

In the invention, a process in which a first color portion of the wax cube (injection molded article) is integrally formed by injecting a first-color colored wax into a die with pressure from an injection nozzle under the condition that rod-shaped core pins (pieces) provided on a slide core is inserted into the die as mentioned later is referred to as ‘injection molding process’, a process in which a second-color colored wax is injected into the empty space of an injection molded article (work in process) consisted of only the first color portion in the die to fill the empty space with the second-color colored wax is referred to as ‘injection process’, and a process including the injection molding process or the injection process is referred to as ‘molding process’.

When the production of the colored wax and the molding process are performed as a continuous process in producing the wax cube, the colored wax which is discharged from the wax-mixing and stirring apparatus described above and controlled at temperatures of about 40° C. to 45° C. is temporarily retained in a molding material retention apparatus (hopper) in the injection molding apparatus. In the retention apparatus, a stirring device is provided inside and the stirring of the colored wax is continuously performed, if required. When the injection molding process and the injection process are performed in future after the colored wax is produced, it is necessary to temporarily solidify the colored wax discharged from the wax-mixing and stirring apparatus. In this case, the colored wax is preferably formed in a powder or pellet having about 1 to 2 mm in diameter. In the specification, unless otherwise noted, the powder or powdering shall include the pellet or palletizing. When the colored wax is powdered, in performing the injection molding process or the injection process in future, the powdered colored wax is loaded as a raw material for molding or injection into the hopper in the injection molding apparatus. Then the powdered colored wax is supplied to the heated filling cylinder and the injection molding or the injection can be easily conducted with the colored wax in the same manner as the conventional plastic injection molding. When the colored wax is maintained in the liquid state without stirring, there is a problem that the pigment in the crayon pastel used as the coloring agent is precipitated to generate color heterogeneity in producing the wax cube. When the colored wax is maintained as a large block in a solid state, there is required in using the colored wax again that the same process as the aforementioned process of producing the colored wax will be conducted, that is, the whole colored wax is melted in the vessel and then the resulting liquid colored wax is controlled at temperatures of about 40 to 45° C. Thus, the powdering of the colored wax has an advantage as means for preserving the colored wax for a long time under the condition as the coloring agent is evenly dispersed in the wax. The powdering of the colored wax has also the advantage that handling of the colored wax becomes easy in the process of forming the wax cube (in injection molding process and the injection process) in future. So a method for producing the colored wax powder will be described before the description of a process for producing the wax cube.

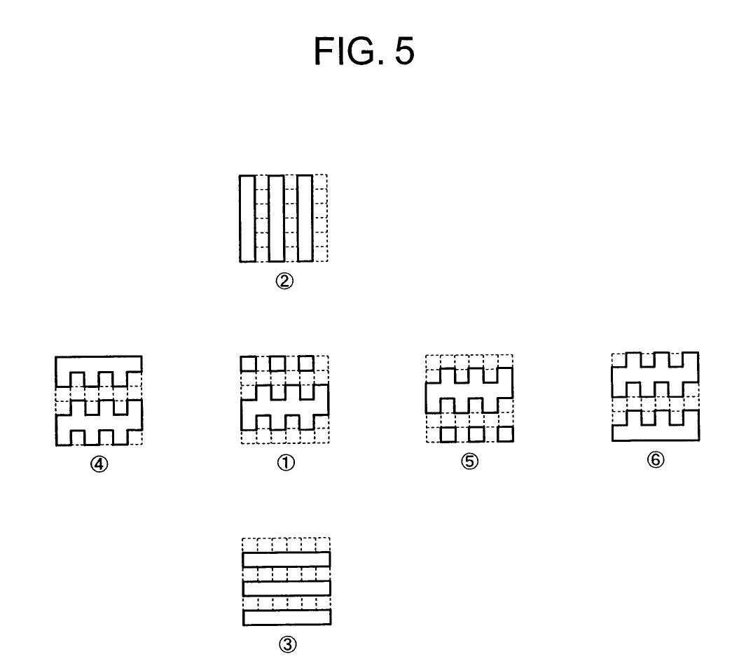

In the method for powdering above-mentioned colored wax whose temperature is controlled in the range from about 40 to 45° C., there can be used proper methods and apparatuses such that the melted colored wax is extruded in a linear shape by an extruder and then the linear-shaped colored wax is finely cut. A preferred example of the method and apparatus for powdering the colored wax with the extruder will be described referring to FIG. 4. A powdering apparatus 40 shown in FIG. 4 includes a material input port 41 that also acts as the retention portion for the melted and stirred material, a wax extruder 42, an extrusion screw 43 that is arranged in the wax extruder 42, a mouth piece 45, a perforated die 46 connected to the mouth piece 45, in the peripheral surface of which many holes 47 are made, and a grinding net 48 that is oscillatably installed on an outer peripheral surface of the perforated die 46. The colored wax transported from the wax-melting and stirring apparatus 20 through the material-transporting pipe 29 is provided to the material input port 41 of the powdering apparatus 40, if necessary, and the colored wax is introduced into the wax extruder 42. At this point, the temperature of the material input port 41 is held at a moderate temperature such that the melted and stirred material is not solidified in the material input port 41. The colored wax introduced into the extruder 42 from the material input port 41 is transported toward the mouthpiece 45 by the extrusion screw 43. A band heater 44 as the heating device is wound around the wax extruder 42 to control the temperature inside the wax extruder 42, keep the softened state of the colored wax in the wax extruder 42, and not solidify therein. The colored wax extruded through the mouthpiece 45 by the extrusion screw 43 is discharged though the holes 47 of the perforated die 46 connected to the mouthpiece 45 and the pulverizing net 48 provided in the outer peripheral surface of the holes. At this point, the solidified colored wax falls down in the form of the micro pellet having about 1 to 2 mm in diameter by giving sieve oscillation to the pulverizing net 48. Even if the coloring degrees of powdered waxes are partially different, a powdered colored wax 49 is homogenized as a whole in use, so that the powdered colored wax 49 has the advantage that the color heterogeneity of the product after molding of the wax can be prevented.

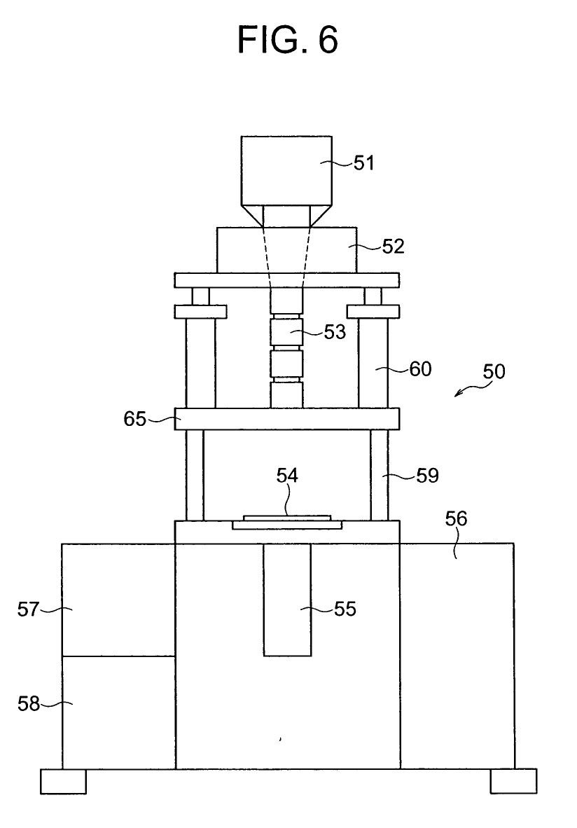

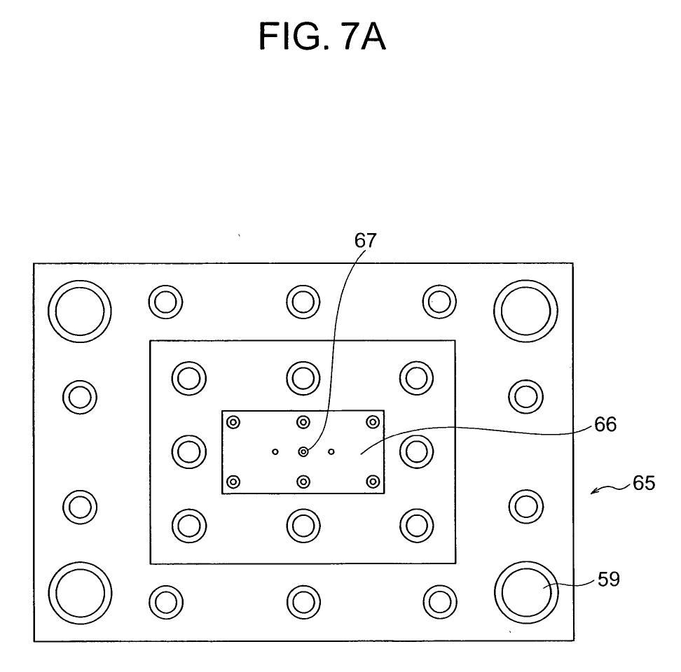

Next, a method of integrally forming the first-color wax portion of the wax cube, e.g. the green work-in-process wax cube by an injection molding using the powdered colored wax or using directly the aforementioned colored wax discharged from the wax-melting and stirring apparatus as the raw material will be described below. FIG. 5 is a development view (hexahedral view) of the green work-in-process wax cube with a pseudo-checkered pattern, which is obtained by the injection molding. FIGS. 6 to 10 show examples of an injection molding apparatus and a die that are used for the injection molding of the wax. FIG. 6 is a schematic outline view of a vertical injection molding apparatus. In FIG. 6, an injection molding apparatus 50 includes a material supply port (hopper) 51, a screw drive motor 52, a filling cylinder 53, a backing plate (jig attaching platen) 54, an ejector mechanism 55, a working condition control board 56, a working condition control terminal panel 57, a main drive-motor 58, a tie bar 59, and a die clamping cylinder 60. The die (core portion) for producing the work-in-process wax cube is attached to the backing plate 54.

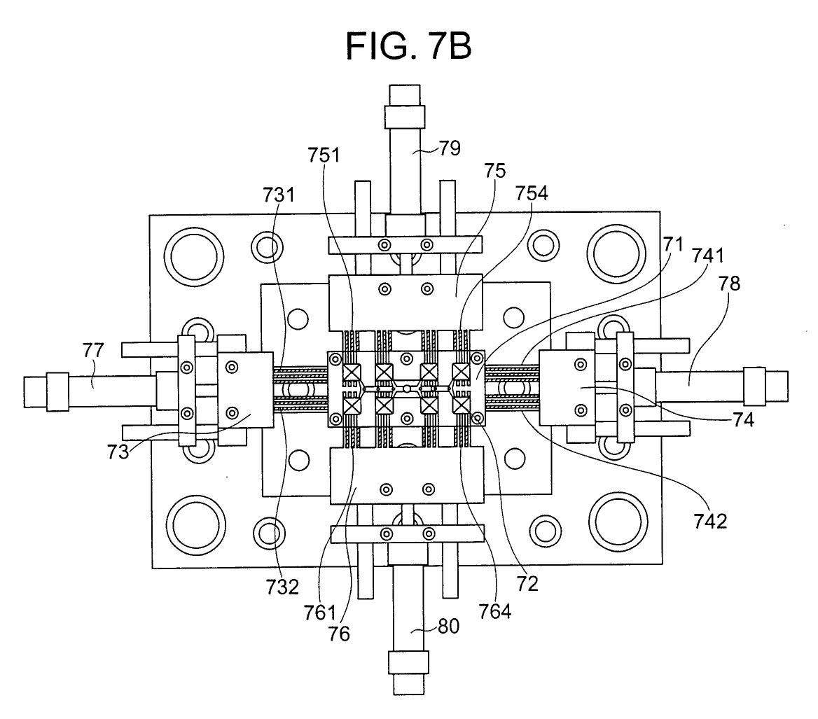

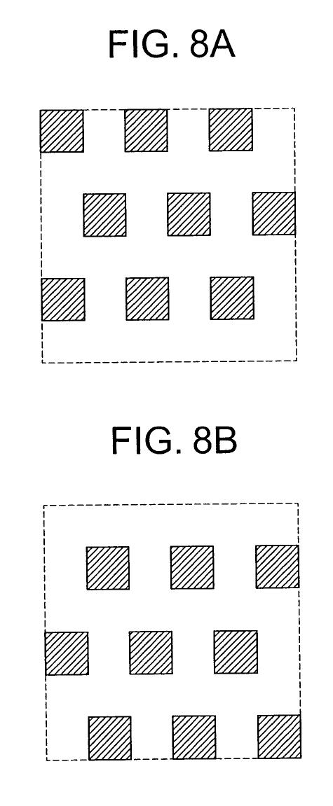

In FIGS. 7A and 7B, an example of the die for producing the work-in-process wax cube is showed. FIG. 7A is a bottom view of a die on a movable die plate (cavity) side, and FIG. 7B is a plan view of the die on a fixed die plate (back plate) side. In FIG. 7A, reference numeral 65 represents a movable die plate, 66 represents an upper surface portion of a parting face of the die, and 67 represents a sprue. In FIG. 7B, reference numeral 71 represents a die (core) on a cavity side, in which eight cubic concave portions 72 having the dimensions of 12 mm×12 mm×12 mm are formed in two lines along a lengthwise direction. In side faces of the core 71, each set of nine holes having the sectional dimensions of about 2 mm×2 mm is provided at a position corresponding to each cubic concave portion. In the side faces in the lengthwise direction, each set of the nine holes is arranged as shown in FIG. 8A. On the other hand, as shown in FIG. 8B, the arrangement of each set of the nine holes in the direction orthogonal to the side face in the lengthwise direction differs from that of the nine holes shown in FIG. 8A in the position. A slide core pin (piece) is movably to be inserted into each hole. Each slide core pin is formed by a rod-shaped member having a cross-sectional area of about 2 mm×2 mm. The nine slide core pins form a set of slide core pins, in which three slide core pins are arranged in line at about 2 mm intervals in a crosswise direction and the three stairs of the three slide core pins are arranged at about 2 mm intervals in a longitudinal direction. The two sets of slide core pins 731 and 732 or 741, and 742 are provided in each side face orthogonal to the lengthwise direction of the core 71 (horizontal direction of FIG. 7B). Further the four sets of slide core pins 751 to 754 or 761 to 764 are provided in each side face in the lengthwise direction of the core 71 (vertical direction of FIG. 7B). These slide core pins are attached to core pin attaching portions 73 to 76 of the slide core. These slide core pins are used in order to form the empty space, to which the red wax is injected later, in the green work-in-process wax cube, so that each slide core pin has at least a length that pierces through the concave portion 72 or a length that reaches a wall surface of the concave portion opposing to a leading edge face of the core pin.

In the example showed in FIG. 7, each set of the slide core pins 731, 732, 741 and 742 in the lengthwise direction of the core 71 has the length so as to pierce through the empty spaces of two adjacent concave portions 72 located in the lengthwise direction of the core 71, and each set of the slide core pins 751 to 754 and 761 to 764 in a width direction has the length so as to pierce through at least the empty space of one concave portion 72. The size of the concave portion 72 of the core 71, the size of the cross section of the slide core and the like are determined in consideration of the reduction of the size caused by compression of the molded article by cooling after the injection molding of the wax.

Hereinafter there is illustrated an example of a method of molding the green work-in-process wax cube using the die of FIG. 7. First the green colored powdery wax previously produced or the colored wax discharged from the wax mixing and stirring apparatus is put into the material supply port (hopper) 51 of FIG. 6. Then, the colored wax in the hopper is sequentially transported into the filling cylinder 53. The filling cylinder 53 is heated from the outside to maintain at the proper viscosity suitable for the injection in the filling cylinder, and the colored wax is stirred by the drive of the screw in the cylinder. On the other hand, the die plate on movable side (movable die plate) 65 is made to fall in order to clamp the die, and after clamping the die the wax is injected into the die to fill the die with the wax by the filling cylinder 53. The temperature in the filling cylinder of the injection molding apparatus is set so as to maintain the viscosity in which the colored wax can be injected into the die and the colored wax is not leaked through the gap of the die. The wax injection molding differs largely from the conventional plastic injection molding at the points where the size of the molded wax article is compressed as the temperature of the molded wax article is lowered and the injection temperature is considerably low. Although the wax temperature depends on the material used, usually it is preferable that the wax temperature is set to the range from about 40 to about 45° C. In addition, it is preferable to set the injection pressure, for example, to the range from about 3 to 6 kgf/cm², and usually to about 4 kgf/cm² although injection pressure depends on injection temperature, the wax used and the like. In the injection molding, the colored wax is injected into each die through the sprue, the runner, and the gate to fill the die with the colored wax. After the injection of the wax is completed, the die is released by lifting the movable die plate. At this point, it is possible that the slide core is controlled so as to back (the slide core pins are pulled out from the core concave portion) as the movable die plate is lifted. It is also possible that the slide core is controlled so as to back after the movable die plate is lifted and then the injected wax is cooled. Further it is also possible that the movable die plate is lifted after the wax is cooled. The movement and the control of the slide cores are performed by air cylinders 77 to 80 attached to the core pin attaching portions 73 to 76 of the slide core. Then, the ejector mechanism 55 ejects the work in process. Unnecessary portions such as the runner, the gate, and the burr are removed from the ejected injection molded article (work in process) to form the eight work-in-process wax cubes (see FIG. 5 for the shape). When the work-in-process wax cube is preserved until the next process, it is preferable that the work-in-process wax cube is preserved in a sterilized apparatus. The sectional views of the groups of core pins in the lengthwise direction and in the crosswise direction, e.g. the sectional views of the core pin groups 751 and 731 are same as ones shown in FIGS. 8A and 8B. In the die concave portion 72, the core pin groups 751 and 731 are made in the different stages each other to construct the pseudo-checkered pattern.

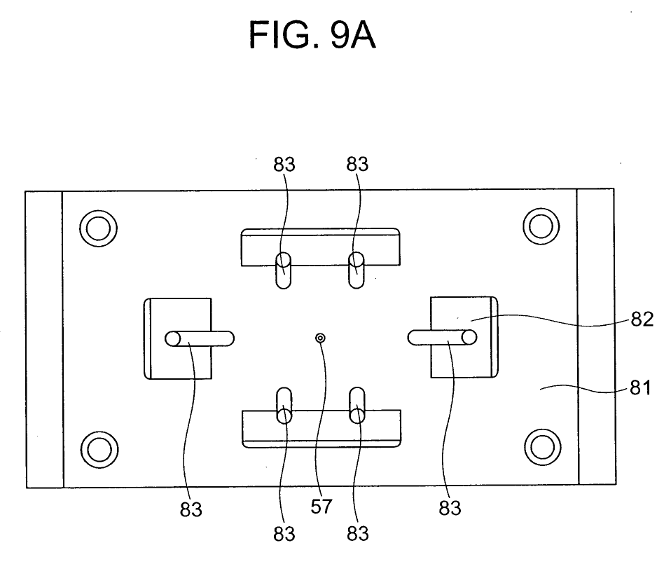

In the above embodiment, the push or pull control of the slide cores are performed by air cylinders. Then, an example of the case in which the push or pull control of the slide core is conducted by an angular pin will be described below referring to FIGS. 9A and 9B. FIG. 9A is a bottom view of a die plate 81 on movable side (movable die plate). In FIG. 9A, on core plates 82, angular pins 83 that diagonally stand toward the insides of the core plates 82 are provided corresponding to the slide cores in the movable die plate 81. On the other hand, FIG. 9B is a plan view of the die plate on the fixed side. As with FIG. 7B, the core pins are inserted into the concave portion 72 of the core 71 through the side face of the core 71. A circular hole 84 into which the angular pin is put is diagonally made in each of the core pin attaching portions 73 to 76 of the slide cores. Therefore, the angular pin 83 falls accordance with the fall of the movable die plate 81. As the angular pins 83 falls, each of the circular holes 84 in the core pin attaching portions 73 to 76 of the slide cores is pressed toward the direction in which the slide cores pins are inserted into the concave portions of the die, i.e. the direction in which the core pin attaching portions 73 to 76 of the slide cores are moved toward the central portion of the core 71. At the time when the die clamping is completed, the core pin reaches at least the opposing wall surface in the concave portion 72, and the injection of the colored wax into the concave portion of the die is enabled. In this state of things, the colored wax is injected into the concave portion of the die. When the movable die plate is lifted after a predetermined time elapses, the core pin attaching portions 73 to 76 of the slide cores are moved along a core guide 85 from the central portion of the die toward the outside. Then, the ejector mechanism 55 ejects the work in process. As with the example shown in FIG. 7B, the unnecessary portions such as the runner, the gate, and the burr are removed from the ejected injection molded article to form eight work-in-process wax cubes.

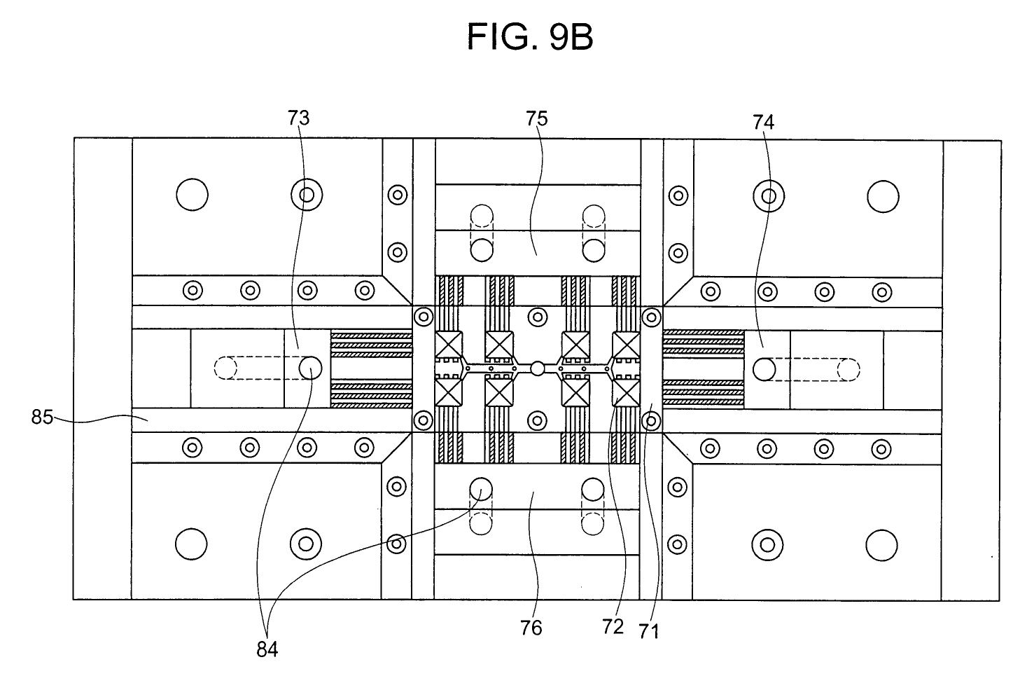

The empty space that is the place where the slide cores are pulled out exists in the work-in-process wax cube produced in the above-described way. Then, the empty space corresponding to the unfilled portion (red wax filling portion) in the work-in-process wax cube is filled with the second-color red wax to form a finished product. A method of filling the empty space with the red colored wax according to the invention will be described referring to FIG. 10. Basically the injection and the filling of the red colored wax can be performed using the same injection molding apparatus shown in FIG. 6 except that the structure of the die plate on the fixed side is different. Therefore, the description of the movable die plate for second-color, which has the same structure as in FIG. 6, will be omitted. FIG. 10 is a plan view of the die plate on the fixed side. In FIG. 10, a jig 92 in which eight work-in-process wax cubes can be positioned is fixed in the central portion of a backing plate 91. The jig 92 has side-face forming portions 921 and 922 that form the two side faces of each wax cube in the eight work-in-process wax cube positioning portions thereof. A runner 98 and a gate 99 are formed in the upper surface of the jig 92. Sliders 93 and 95 are attached to the backing plate 91 as the sliders 93 and 95 can slides on the upper surface of the backing plate 91 in the direction of the jig 92 by air cylinders 94 and 96. When the sliders 93 and 95 are pressed against the jig 92, the surfaces facing to the jig 92 in the sliders 93 and 95 have other side-face forming portions 931 and 932 or 951 and 952 that form the remaining two sides of each wax cube. Accordingly, when the sliders 93 and 95 are pressed against the jig 92, the sliders 93 and 95 and the jig 92 can form a concave portion 97 having the size of eight wax cubes. In the filling of the red colored wax, the work-in-process wax cubes are loaded in a given run or direction so that the empty space of the work-in-process article faces the gate, while the eight work-in-process wax cubes are in close contact with the side faces 921 and 922 for positioning of the jig 92. Then, the slider 93 and 95 are pressed by the air cylinders 94 and 96 to be in close contact with the jig 92, as the jig 92 and the sliders 93 and 95 surround the green work-in-process wax cube. It is also possible that the work-in-process wax cubes are put into the eight concave portions 97 that are formed after the jig 92 comes into close contact with the sliders 93 and 95. Subsequently, the movable die plate falls. When the jig 92 and the sliders 93 and 95 are pressed against the movable die plate to complete the die clamping, the red colored wax is injected into the die through the sprue. After the injection is ended, the movable die plate is lifted, and the gate portion of molded wax is cut if needed. Then, the sliders 93 and 95 are backed to take out the injected product, and the burr and the like are further removed to form the finished product. Alternatively, the ejector mechanism ejects the injection-molded product to take out the injection-molded product before the sliders 93 and 95 are backed, and thereafter the unnecessary runner, gate, and burr are removed to form the finished product.

For the injection temperature and the injection pressure in injecting the red colored wax into the work-in-process wax cube, the same temperature and pressure as the green colored wax can be adapted in the injection molding. In the embodiment described above, the work-in-process wax cube is first produced using the green colored wax, and then the red colored wax is injected into the empty space of the green work-in-process wax cube to fill the empty space with the red colored wax to form the finished product. However, needless to say, it is possible that the work-in-process wax cube is first formed using the red colored wax and then the green colored wax is injected into the empty space to fill the empty space with the green colored wax to form the finished product.

In the wax cube producing method of the preferred embodiment described above, the core pins are configured to be automatically inserted into or pull out from the concave portions 72. However, the core pins in the lengthwise direction and the crosswise direction may be inserted into and pull out from the concave portions 72 by hand control. In this case, the core 71 having the eight concave portions and made a set of nine holes, through which rod-shaped members (pieces) corresponding to the core pin are inserted, on the side face and if needed in a partition wall between the concave portions is fixed to the backing plate. The rod-shape member is then manually inserted into each hole, and the rod-shaped member of the slide core pierces through each concave portion. Thereafter, the backing plate can be lifted to press the core 71 against the fixed die plate, and the colored wax is injected to fill the concave portion in the pressed state. Alternatively portions corresponding to the core pins in the lengthwise direction or crosswise direction may be previously formed in the die. In this case, the remaining core pins are inserted through the holes made in the side face of the die, the inserted core pins and the core pins previously formed in the die are arranged in the pseudo-checkered pattern, the rod-shape members are extracted after the wax is injected, and the work-in-process wax cube can be taken out by opening the core of the die in the center.

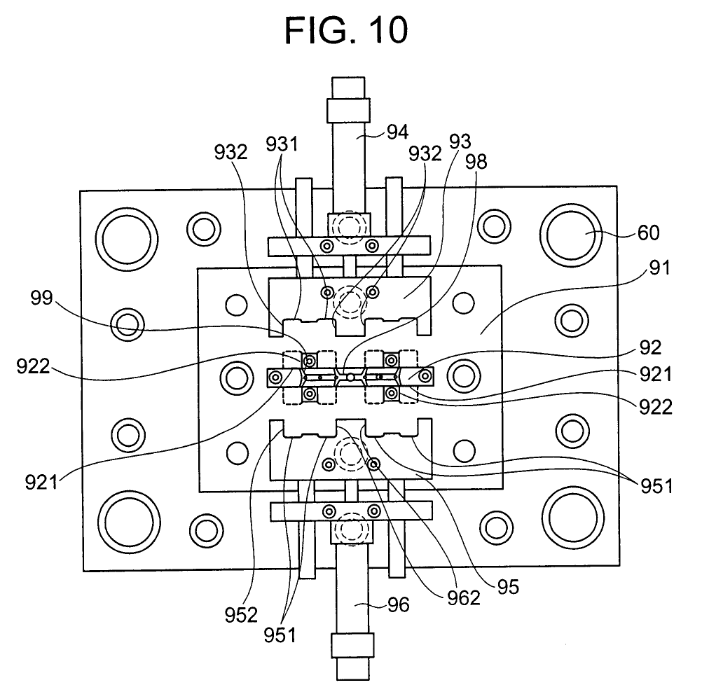

In the embodiments above, the injection molding process of the first-color colored wax and the injection process of the second-color colored wax are conducted in the different process, that is, the work-in-process wax cube is temporarily formed by injecting the first-color colored wax into the die, then the core pins are pulled out from the concave portion 72 to take out the work-in-process wax cube from the die. The runner and the burr are removed from the work-in-process wax cube, and the second-color colored wax is injected into the empty space of the work-in-process wax cube to form the finished product. However, after the work-in-process wax cube is formed with the first-color colored wax, the wax cube may continuously be produced by injecting the second-color colored wax into the same die without taking out the work-in-process wax cube from the die, if necessary. An example of a method and apparatus for continuously producing the wax cube according to the invention will be described below referring to FIGS. 11 to 13.

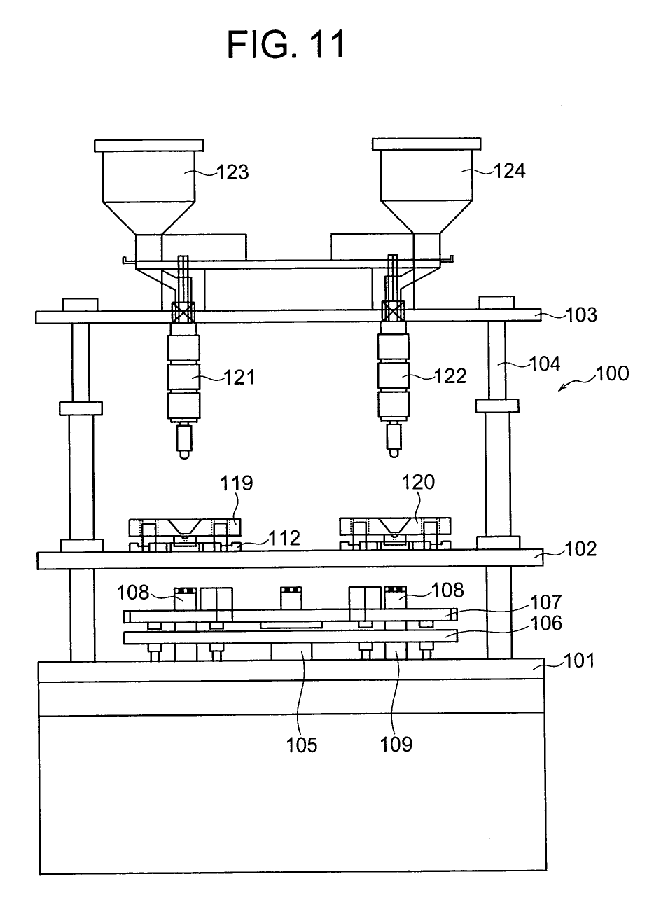

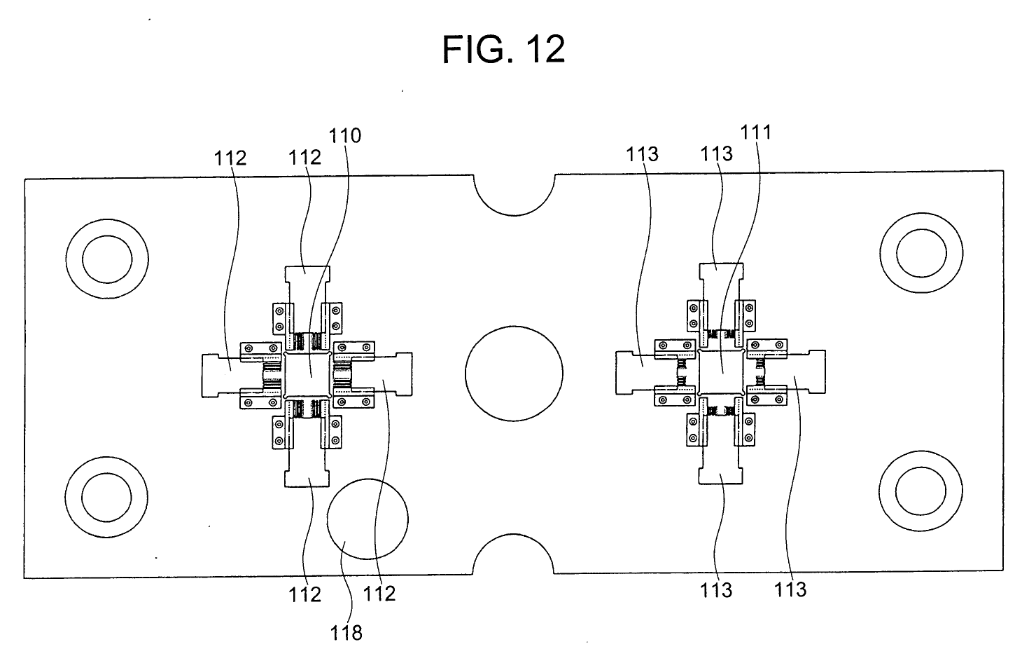

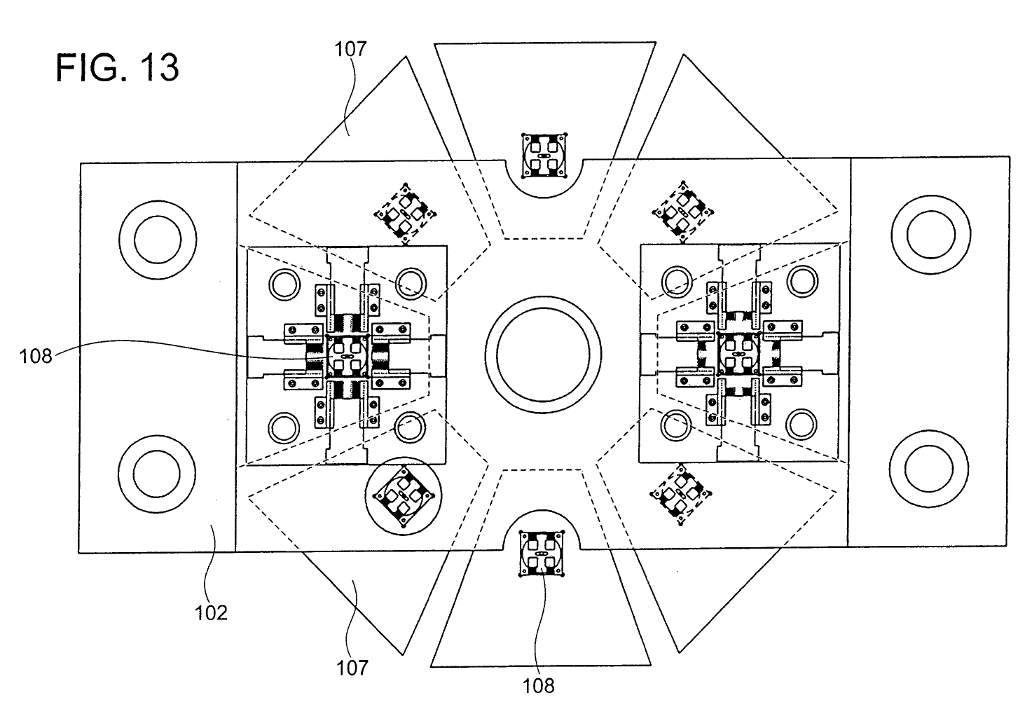

FIG. 11 is a front view of an apparatus 100 for producing continuously and automatically a masticatory function test wax cube. The apparatus 100 includes a base 101, a fixed plate 102 fixed to the base 101, and a movable plate 103 attached vertical-movably by corner cylinders 104 over the fixed plate 102. Between the base 101 and the fixed plate 102, a rotating plate 106 and die fixing plate 107 constituted by eight plates on the rotating plate 106 are provided. The rotating plate 106 is attached to a rotating shaft 105 and the die fixing plates 107 are rotated with the rotating plate 106 when the die fixing plates 107 are positioned on the rotating plate 106, and the die fixing plates 107 can be lifted when a pushing-up rod 109 pushes up the die fixing plates 107 through the rotating plate 106. Further dies (cores) 108 is fixed to each die fixing plate 107. In FIG. 11, for purposes of illustration, the die fixing plates 107 are somewhat floated from the rotating plate 106. FIG. 12 is a plan view of the fixed plate 102 of the apparatus 100 shown in FIG. 11, FIG. 13 shows the shape of the die fixing plates 107 and a positional relationship between the fixed plate 102 and the die fixing plates 107, and FIG. 14 is an enlarged view showing the slide core arranging portion of the fixed plate 102. FIG. 14A shows the first-color colored wax filling portion, and FIG. 14B shows the second-color colored wax filling portion.

As shown in FIG. 12, square holes 110 and 111 are made at two points in the fixed plate 102. Slide cores 112 and 113 are provided in the four directions of the holes 110 and 111 and around the holes 110 and 111 respectively. The side cores 112 and 113 can be slid toward the four directions of the holes 110 respectively, and the core pins are fixed to the slide cores 112 and 113 respectively. As shown in FIGS. 11 and 13, the die (core) 108 is fixed onto the die fixing plates 107 located below each of the holes 110 and 111. The dies 108 can be inserted into the holes 110 and 111 respectively by lifting the die fixing plates 107 located below the holes 110 and 111. Upper plates 119 and 120 of the dies 108 are fixed to the upper portions of the holes 110 and 111 so as to cover the holes and the dies 108 are clamped by lifting the die fixing plates 107. In the die (core) 108, four concave portions for producing the wax cubes are formed on the upper surface and in the same plane. As described in explanation of FIGS. 7A and 7B, a set of nine holes is made in each side face of the concave portion, and the two sets of nine slide core pins are inserted into each of the concave portions in the different stages from two directions so that the two sets of nine slide core pins arranged in the different stages each other to construct the pseudo-checkered pattern after clamping the die. Filling cylinders 121 and 122 are attached to the movable plate 103 at the opposite positions to the holes 110 and 111, and material supply ports (hopper) 123 and 124 are connected to the filling cylinders 121 and 122 respectively. After the slide core pins are inserted, the movable plate 103 falls, and the first-color colored wax and the second-color colored wax are simultaneously injected into the dies 108 and 108 from the filling cylinders 121 and 122 through die upper plates 119 and 120, respectively. In this case, the left side portion (hole 110) of FIG. 11 is defined as the first-color colored wax injecting portion, and the right side portion (hole 111) is defined as the second-color colored wax injecting portion. In the first-color die, the leading end of each slide core pin is inserted till reaching to the opposing surface of the core concave portion or to the position where the leading end of the slide core pin pierces through the opposing surface of the core concave portion by an un-shown pushing apparatus. On the other hand, in the second-color die, the insertion depth is controlled so that the leading end surface of the slide core pin reaches the position where the leading end surface forms the same plane as the inner wall surface of the concave portion. At this point, as shown in FIGS. 14A and 14B, core pins 114 for the first-color are formed to be longer than core pins 115 for the second-color by a predetermined length, which allows the leading end surfaces of the slide core pins to be controlled so as to reach the same positions described above, even if the core pins 114 for the first-color and the core pins 115 for the second-color are moved to the same distance. After the colored wax is injected into each die, the filling cylinders 121 and 122 are lifted together with the movable plate 102, the slide core pins are pulled out from each die, and the die 108 is lowered. Then, the die fixing plates 107 are lowered to the position of the rotating plate 106 and located at the predetermined position of the rotating plate. And then the die fixing plates 107 are rotated by 45° as the rotation of the rotating plates 106 and stopped. In addition, when the die fixing plates 107 located below the holes 110 and 111 in the fixed plate 102 are lifted, the object of the invention can be achieved. However, in this apparatus, the die fixing plate (on which cooled finished product exists in the die) located at a front position in the rotating direction of the first-color injecting position is also lifted at the same time, the upper portion of the die is projected from a circular hole 118 in the fixed plate 102, the masticatory function test wax cube is ejected by the ejector mechanism ejects at this position, and the wax cube is collected.

In the apparatus 100 for producing continuously and automatically the masticatory function test wax cube shown in FIG. 11, the rotating plate 106 and the die fixing plates 107 are separately provided. However, it is possible that the rotating plate and the die fixing plates are integrally provided and the rotating plate to which the die is fixed is lifted as a whole after the die fixing plates are rotated by the predetermined angle. In this case, it is necessary that the holes are provided respectively at places corresponding to the die lifting positions in the fixed plate so that the die does not hit against the fixed plate.

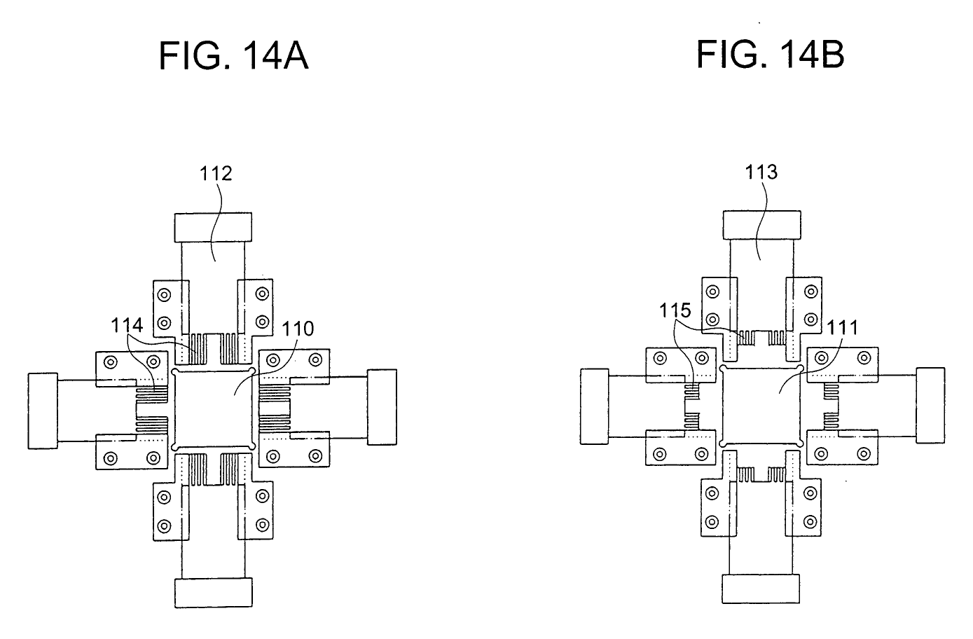

Another example of an apparatus for producing continuously and automatically a masticatory function test wax cube will be described referring to FIG. 15. FIG. 15 is a plan view of an apparatus 130 for producing continuously and automatically a masticatory function test wax cube. Unlike the above-described injection molding apparatuses, the injection apparatuses of this example are horizontally arranged in parallel. In FIG. 15, reference numeral 131 represents a first-color colored wax injecting apparatus, and reference numeral 132 represents a second-color colored wax injecting apparatus. In the apparatus 130, one die is used. In the die, one concave portion for producing one wax cube is provided. And the die (core) is provided on the movable side. A set of nine core pins is arranged around the die on the movable side while the set of nine core pins can be inserted into and pulled out from the concave portion of the die in four directions of the die. The die provided on the movable side is clamped by pressing the die against the die on the fixed side at the first-color injecting position. At this point, the slide core pins are inserted into the concave portion of the die by the action of the angular pin provided in the die on the fixed side, the slide core pins are arranged in the concave portion in the different stages each other so as to construct the pseudo-checkered pattern, and the first-color colored wax is injected into the die in this state of things. After the die is opened, the die is conveyed to the second-color colored wax injecting position. At this point, the core pins are backed from the concave portion of the die. After the die is located at the second-color colored wax injecting position, the die is clamped again. The leading end of the slide core pin is inserted to the position where the leading end partially forms the inner wall surface of the concave portion of the die by the angular pin provided in the fixed side die of the second-color colored wax injecting apparatus, and the second-color colored wax is injected. Then, the die is opened, the ejector mechanism ejects the wax cube after the cooling, the wax cube is collected, and the die is conveyed to the first-color colored wax injecting position 2.

Alternatively, in the apparatus of FIG. 15, the following method can be also adopted. That is, the die is formed so that the slide core pin attaching wall surface constitutes the inner wall surface, the slide core pin is moved to the central portion by the action of the angular pin provided in the die on the fixed side in clamping the die to form the concave portion for forming a wax cube by the four surfaces of the slide core pin attaching wall on the slide core, and the pseudo-checkered pattern unfilled portions (portion to be filled with the second-color colored wax) in the different stages by the slide core pin. When the die is opened after injecting the first-color colored wax into the die, the slide core is backed away from the wax cube so that only the work-in-process wax cube remains at the forming position. In the state of things, the work in process and the slide core are conveyed to the second-color colored wax injecting position. In the second-color colored wax injecting portion, a die is attached to a fixed-side attaching plate 133. The die has the concave portion whose size is same as of the wax cube and has a wall having thinner thickness than the backed distance of the slide core from the work-in-process wax cube. When the die clamping is performed by the movement of a die 134 on the movable side, the work-in-process wax cube that remains in the die on the movable side is housed in the concave portion above, and the second-color colored wax is injected into the concave portion of the die from a filling cylinder 135 after the die clamping.

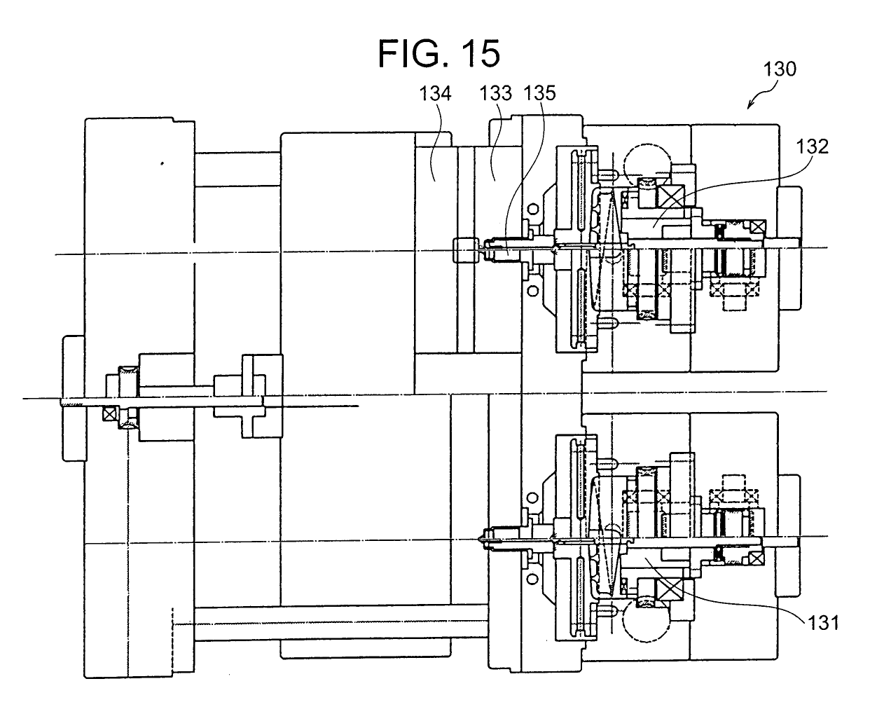

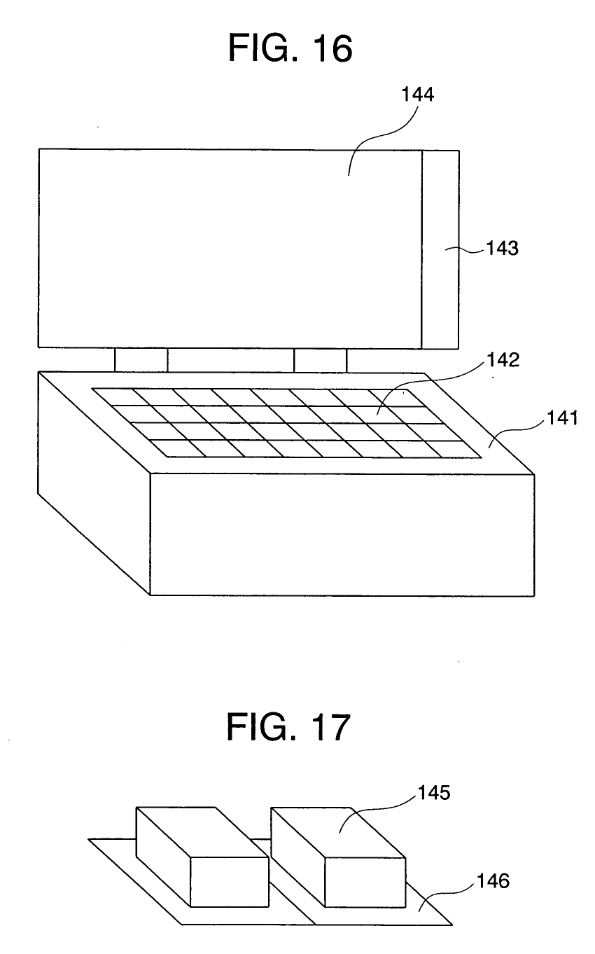

From the viewpoint of quality preservation in distribution and inventory management, it is preferable that the wax cube of the finished product is packaged one by one. With reference to the packaging work, for example, it is preferable to adopt the method of using an apparatus shown in FIG. 16. In FIG. 16, a backing sheet for packaging is put on an input portion 142 of a frame 141, and each wax cube is loaded in each concave portion of the backing sheet. Then, the packing film is pulled out from a packing film roll 143, hot bonding is performed by lowering a blister sealer 144 with a hot plate for bonding, and the wax cube is blocked from the atmosphere by seal. Therefore, as shown in FIG. 17, each wax cube is packaged in each concave portion of the backing sheet 145 to form the package in which the wax cube is sealed with a packing film 146.

In the aforementioned production of the colored wax, the colored wax is produced with the wax-melting and stirring apparatus. However, it is also possible to adopt a method in which the wax and the coloring agent are put into the vessel such a pot, the vessel is heated by putting the vessel in hot water, the wax and the coloring agent are heated to melt the wax, and the wax and the coloring agent are sufficiently mixed together to produce the even colored wax solution by stirring the melting solution.

When compared with the conventional method and apparatus for producing the wax cube, the high-quality masticatory function test wax cube can evenly be produced with high efficiency and without generating the defective product by the method and apparatus according to the invention. In the masticatory function test wax cube according to the invention, the two-color waxes are in close contact with each other with no gap, and two colored waxes are evenly connected to each other with sufficient strength, so that the masticatory function test wax cube can preferably used for the masticatory function test.

Thus, the method and apparatus for producing the masticatory function test wax cube according to the invention are described in detail on the basis of the specific apparatus. However, the invention is not limited to the methods and apparatuses specifically described above, and it is understood that various changes and modifications may be made without departing from the spirit and scope of the invention.

1. A method for producing a two-color masticatory function test wax cube with a pseudo-checkered pattern used for evaluating a masticatory performance, wherein a first-color colored wax is injection-molded to produce integrally a first-color wax portion of the wax cube and then a second-color colored wax is injected and filled into empty space of the wax cube to produce a wax cube consisting of two-color colored waxes with a two-color pseudo-checkered pattern.

2. A method for producing a masticatory function test wax cube according to claim 1, wherein the colored wax constructing the wax cube comprises a wax and a coloring agent.

3. A method for producing a masticatory function test wax cube according to claim 2, wherein the wax comprises a paraffin wax and another wax that imparts adhesive properties to the paraffin wax, and the coloring agent comprises a crayon pastel.

4. A method for producing a masticatory function test wax cube according to any one of claims 1 to 3, wherein colored wax of a raw material used for the injection molding or injection is supplied in powder form to an injection molding apparatus or an injection apparatus.

5. A method for producing a masticatory function test wax cube according to any one of claims 1 to 3, wherein the colored wax of a raw material used for the injection molding or injection is produced as a homogeneous colored wax solution by melting and mixing a wax and a coloring agent and then supplied to an injection molding apparatus or an injection apparatus in the condition cooled down to 40° C. to 50°.

6. A method for producing a masticatory function test wax cube according to any one of claims 1 to 3, wherein an injection molding temperature or an injection temperature of the colored wax ranges from 40° C. to 45° C.

7. A method for producing a masticatory function test wax cube according to any one of claims 1 to 3, wherein, when a first-color injection molded article is produced with the colored wax, a slide core is arranged around the molding die to produce the injection molded article having a multi-stage structural pseudo-checkered pattern.

8. A method for producing a masticatory function test wax cube according to claim 7, wherein the first-color injection molded article that is a work-in-process wax cube is produced and then taken out from a first molding die and after the first-color injection molded article is placed into a second molding die, the second-color colored wax is injected into the empty space of the first-color injection molded article housed in the second molding die to produce a wax cube with a two-color pseudo-checkered pattern.

9. A method for producing a masticatory function test wax cube according to claim 7, wherein after the first-color injection molded article that is a work-in-process wax cube is produced, a slide core pin is pull out from the inside of the molding die without taking out the first-color injection molded article from the molding die, and then the second-color colored wax is injected into the empty space of the first-color injection molded article in the molding die to produce a wax cube with a two-color pseudo-checkered pattern.

10. A method for producing a masticatory function test wax cube according to claim 7, wherein a die is formed by moving of four slide cores having slide core pins in clamping the die, a first-color injection molded article is produced by injecting a first-color colored wax into the die, the slide cores are backed by a die opening operation, then the first-color injection molded article is moved together with the slide core to a second-color molding portion, the first-color injection molded article is housed in a second die in which the injection molded article can be housed, and second-color colored wax is injected into an empty space of the first-color injection molded article to produce a wax cube with a two-color pseudo-checkered pattern.

11. A method for producing a masticatory function test wax cube according to any one of claims 1 to 3, wherein the masticatory function test wax cubes including two-color colored waxes are placed in each concave portion of a backing sheet for packing, and the each concave portion is sealed with a package film.

12. An apparatus for producing a first-color injection molded article of a masticatory function test wax cube, comprising;
nine substantially square holes which are pierced through a side wall of a die having outside surfaces orthogonal to each other from the outside surfaces of the die toward the cubic concavity in the die, the nine square holes being made in each face,
a slide core provided with nine rod-shape core pins having a substantially square cross-sectional area so as to pierce through the holes,
a slide core-moving device for moving the slide core, and
an injection nozzle for injecting a first-color colored wax into a concavity of the die.

13. An apparatus for producing a first-color injection molded article of a masticatory function test wax cube according to claim 12, wherein the core pin is attached to a core pin attaching portion of the slide core, and a core pin attaching portion of the slide core can be moved by an air cylinder.

14. An apparatus for producing a first-color injection molded article of a masticatory function test wax cube according to claim 12 or 13, wherein the core pine is attached to a core pin attaching portion of the slide core, and a hole for piecing an angular pin is formed in the core pin attaching portion of each slide core, and the slide core can be moved corresponding to inserting into or drawing out the angular pin with the hole.

15. An apparatus for producing a masticatory function test wax cube, comprising;
a jig fixed to a backing plate, which has a surface for positioning the first-color injection molded article of a masticatory function test wax cube that is a work-in-process wax cube and forming a side face of a concave portion in which the wax cube injection molded article is housed, a runner and a gate,
a slider that forms a concavity, in which the wax cube injection molded article is to be housed, with the jig, and
an injection nozzle that injects a colored wax into the concavity formed by the jig and the slider.

16. An apparatus for producing a masticatory function test wax cube, comprising;
a fixed plate having two square holes, one of which is a position to form a wax cube injection molded article with a first-color colored wax and another of which is an injection position to inject a second-color colored wax into a die,
a die-fixing plate that is provided below the fixed plate, and is able to revolve and position, in the hole, one of dies fixed to the die-fixing plate by moving the die-fixing plate upward,
the dies which are fixed to the die-fixing plate and through side walls of which holes for respectively inserting a slide core pin are opened,
slide cores that are arranged in each side of the hole and have the slide core pins,
slide core moving means for moving the slide core, and
colored wax-filling cylinders that are movably arranged above the each hole.

17. An apparatus for producing a masticatory function test wax cube in which a filling cylinder for injecting a first-color colored wax into a die and a filling cylinder for injecting a second-color colored wax into the die are connected in parallel, wherein
the die has a square shape,
nine holes into which core pins are inserted are made in each side surface of the die,
slide cores in which the core pins are arranged at a position corresponding to each hole into which core pins are inserted, are provided opposing to the side surface of the die,
slide core moving means that cause the core pins to be inserted into the die when the first-color colored wax is injected into the die and cause the core pins to be pulled out from the inside of the die after the injection of the first-color colored wax is completed is provided,
the die is moved to a second-color colored wax injection position by conveying means after the injection of the first-color colored wax is ended, and
a second-color colored wax is injected into the die after a leading end surface of the core pin of the slide core is moved to a position where the leading end face of the core pine forms a part of an inner wall surface of a concavity in the die for forming a wax cube.

18. An apparatus for producing a masticatory function test wax cube in which a filling cylinder for injecting a first-color colored wax into a first die and a filling cylinder for injecting a second-color colored wax into a second die are connected in parallel,
the first die includes four slide cores in which each core pin is formed inward,
core pin-attaching surfaces of the slide cores and core pines are arranged such that a square-shaped concavity is formed by the core pin-attaching surfaces of the slide cores and a multi-stage pseudo-checkered pattern block is formed by the core pins when the slide cores are moved to the center on the same plane,
the first-color colored wax is injected to form a first-color injection molded article for a masticatory function test wax cube (work-in-process wax cube) in the first die while the concavity is formed,
the first die is opened and the core pins are pull out from the inside of the injection molded article for the wax cube,
the injection molded article for the wax cube is conveyed with the first die to a second-color colored wax injection position, and
the masticatory function test wax cube is produced by injecting the second-color colored wax into a cavity portion of the first-color injection molded article, which is formed by pulling out the core pins, by the second-color filling cylinder while the wax cube injection molded article is covered with second die at the second-color colored wax injection position, thus the masticatory function test wax cubes being prepared.

19. A colored wax-powdering apparatus for use in production of a masticatory function test wax cube, wherein the apparatus comprises;
an extruding device for extruding a colored wax,
a perforated die having many holes and being attached to a leading end portion of the extruding device, and
a pulverization net oscillatably attached to an outer peripheral surface of the perforated die.

20. A masticatory function test wax cube including two-color colored waxes, which is formed by injection-molding integrally a first-color colored wax portion, and then injecting a second-color colored wax into an empty space of the wax cube to fill the empty space with the second-color colored wax.
